(19) United States
(12) Patent Application Publication
(10) Pub. No.: US 2020/0019873 A1
(43) Pub. Date: Jan. 16, 2020

(54) System for Choosing Clothing and Related Methods

(71) Applicant: W/You, Inc., Mountain View, CA (US)

(72) Inventors: Anne Miller, Mountain View, CA (US); Brian Martin, Portland, OR (US); Sampriti Bhattacharyya, San Francisco, CA (US)

(21) Appl. No.: 16/513,441

(22) Filed: Jul. 16, 2019

Related U.S. Application Data
(60) Provisional application No. 62/698,793, filed on Jul. 16, 2018.

(51) Int. Cl. G06N 5/04; G06N 5/02; G06F 17/28; G10L 21/10; G16H 10/20; G06Q 30/02; G06N 20/00

(52) U.S. Cl. CPC G06N 5/048; G06N 5/025; G06F 17/2872; G06N 20/00; G16H 10/20; G06Q 30/0282; G10L 21/10

(57) ABSTRACT

Implementations of automated systems for making apparel recommendations may include: a first database having a plurality of apparel items recommended for medical events based on health related criteria. The automated systems may include a second database including two or more questions requesting information about the user. The automated systems may include a natural language processor (NPL) configured to extract semantic primitives from the two or more questions from the free text portion of the user interface. The system may include a third database of one or more retailers of a plurality of apparel items recommended for medical events based on health related criteria. The automated system may include a rules engine configured to use the semantic primitives from the natural language process, the first database, and the third database to produce a personalized list of one or more recommended apparel items for a user who has experienced a medical event.

### CROSS REFERENCE TO RELATED APPLICATIONS

This document claims the benefit of the filing date of U.S. Provisional Patent Application 62/698,793, entitled “Systems for Choosing Clothing and Related Methods” to Anne Miller, which was filed on Jul. 16, 2018, the disclosure of which is hereby incorporated entirely herein by reference.

### BACKGROUND

### 1. Technical Field

Aspects of this document relate generally to automated systems, such as databases that supply information, keep track of various combinations, and employ machine-learning techniques to increase the database. More specific implementations involve a database of expert recommendations for persons dealing with medical challenges and events.

### 2. Background

Conventionally, the process for choosing clothing that is compatible with health-related challenges and health related criteria has been to give a person a checklist to fill out which attempts to address the person's health related criteria. The person relies heavily on the medical professional with whom they are dealing for apparel recommendations. The person may call or email the medical professional repeatedly with apparel related questions.

### SUMMARY

Implementations of automated systems for making apparel recommendations may include: a first database having a plurality of apparel characteristics with each of a plurality of apparel items recommended for medical events based on health related criteria. The automated systems may also include a second database. The second database may include two or more questions requesting information about the user. The two or more questions may be configured to be displayed on a user interface of a computing device. At least one of the questions may be designed to accept a free text response. The automated systems may also include a natural language processor (NPL). The NPL may be configured to extract semantic primitives from the two or more questions from the free text portion of the user interface. The systems may also include a third database of one or more retailers of a plurality of apparel characteristics with each of a plurality of apparel items recommended for medical events based on health related criteria. The automated system may also include a rules engine configured to use the semantic primitives from the natural language process, the first database, and the third database to produce a personalized list of one or more recommended apparel items for a user who has experienced a specific medical event.

Implementations of automated systems may include one, all, or any of the following:

The first database may include apparel items by expert medical recommendations.

The rules engine may include an updating process that continually updates the first database of apparel characteristics with each of a plurality apparel items recommended for medical events based on health related criteria.

The rules engine may use an algorithm including a forward-chaining rules engine that implements a fuzzy logic calculation based on a Bayes' Theorem to produce the personalized list of one or more recommended apparel items.

The personalized list may include recommended apparel items based on one or more criteria including a health challenge of the user, one or more size preferences of the user, one or more color preferences of the user, one or more brand preferences of the users, one or more geographical locations of the user, or any combination thereof. These criteria may be extracted from the two or more answers to the two questions in the user interface.

The natural language processor may be configured to extract semantic primitives from free text responses or voice-to-text transcripts.

Implementations of a database of apparel recommendations may be built using a method for building a database, the method may include: storing, in a first database, a plurality of apparel characteristics with each of a plurality of apparel items recommended for medical events. The recommendations for medical events may be based on information from one or more medical professionals. The method may also include storing, in a second database, two or more questions for a plurality of users. Each user may experience one or more of a plurality of medical events. The method may also include sending, through a telecommunication channel, to a computing device associated with a user, the two or more questions from the second database to the plurality of users. The computing device associated with the user may generate a user interface including the two or more questions in response to receiving the two or more questions. The method may also include receiving from the computing device, through a telecommunication channel, two or more answers to the two or more questions from the user interface. The method may include processing, with a natural language processor, the two or more answers from the plurality of users to extract the one or more medical events of each of the plurality of users and one or more preferences of each of the plurality of users. The method may include generating, using the first database and the rules engine, a list of recommended apparel items for each of the plurality of users based on the one or more medical events extracted from the answers to the two or more questions received from the computing device. The method may include processing, using a third database of apparel retailers and the rules engine, a list of recommended apparel items and the one or more preferences of each of the plurality of users. The method may include generating with the list of the preferred recommended apparel items and the third database of apparel retailers, using one or more calculations of the rules engine, a personalized list of recommended apparel items for each of the plurality of users. The method may include adding, to the first database, the personalized list of recommended apparel items for each of the plurality of users to the first database.

Implementations of methods of building a database may include one, all, or any of the following:

A size of the first database may be increased through machine learning.

The second database may include at least one of a demographic question and free text question.

The rules engine may use an algorithm including a forward-chaining rules engine that implements a fuzzy logic calculation based on Bayes' Theorem to produce the personalized list of one or more recommended apparel items.

The personalized list may include one or more recommended items based on one or more criteria including a health challenge of the user, one or more size preferences of the user, one or more color preferences of the user, one or more brand preferences of the user, one or more geographical locations of the user, or any combination thereof. The one or more criteria may be extracted from the two or more answers to the two questions in the user interface.

The natural language processor may be configured to extract semantic primitives from free text responses or voice-to-text transcripts.

Implementations of personalized lists of apparel recommendations may be generated using an automated method for selecting apparel, the method may include: selecting, a user facing a medical event. The method may also include sending, through a telecommunication channel, a questionnaire to a computing device associated with the user. The computing device may be configured to generate a user interface including the questionnaire through a user interface. The questionnaire may use a second database including two or more questions. The method may include receiving, through a telecommunication channel, two or more answers to the questionnaire from a user via the computing device. The method may include processing, with a natural language processor, the two or more answers from the user to extract one or more medical events of the user and one or more preferences of the user. The method may include generating, using the first database, a list of recommended apparel characteristics with each of a plurality of apparel items for the user using one or more medical events extracted from the two or more answers. The method may include processing, using a rules engine, the list of recommended clothing items and the one or more preferences of the user to form a preferred recommendations list. The method may include generating, using the rules engine and a third database of apparel retailers, a personalized list of recommended apparel items. The method may include sending, using a telecommunication channel, to the computing device the personalized list of items using the computing device generated user interface including a personalized list of recommended apparel items. The method may include sending, using the user interface of the computing device, to one or more preselected potential buyers one or more items from the personalized list.

Implementations of methods of selecting apparel may include one, all, or any of the following:

The user may include one of a person dealing with a medical event, a friend, a family member, a medical professional, a social worker, or any combination thereof.

The rules engine may use an algorithm including a forward-chaining rules engine that implements a fuzzy logic calculation based on a Bayes' Theorem to produce the personalized list of one or more recommended apparel items.

The personalized list may include recommended apparel characteristics based on one or more criteria including a health challenge of the user, one or more size preferences of the user, one or more color preferences of the user, one or more brand preferences of the user, one or more geographical locations of the user, or any combination thereof. The criteria may be extracted from the two or more answers to the two questions in the user interface.

The natural language processor may be configured to extract semantic primitives.

The method may further include sending a beneficiary user of the user a unique identifier of a beneficiary user interface to notify the beneficiary user of the beneficiary user interface.

Sending the beneficiary user a unique identifier may include one of sending an email or sending a postcard.

The method may further include facilitating the purchase of a personalized item through a third database of apparel retailers.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

Implementations will hereinafter be described in conjunction with the appended drawings, where like designations denote like elements, and:

FIG. 1 is an implementation of system for making apparel recommendations;

FIG. 2 is an implementation of a method of building a database of apparel recommendations based on medical events;

FIG. 3 is an implementation of an automated method for selecting apparel based on medical events;

FIG. 4 is a high level example of an implementation of a method for selecting apparel based on medical events;

FIG. 5 is a detailed example of an implementation of a method for selecting apparel based on medical events;

FIG. 6 is another detailed example of an implementation of a method for selecting apparel based on medical events;

FIG. 7 is another detailed example of an implementation of a method for selecting apparel based on medical events; and

FIG. 8 is another detailed example of an implementation of a method for selecting apparel based on medical events.

### DESCRIPTION

This disclosure, its aspects and implementations, are not limited to the specific components, assembly procedures or method elements disclosed herein. Many additional components, assembly procedures and/or method elements known in the art consistent with the intended system for choosing apparel will become apparent for use with particular implementations from this disclosure. Accordingly, for example, although particular implementations are disclosed, such implementations and implementing components may comprise any shape, size, style, type, model, version, measurement, concentration, material, quantity, method element, step, and/or the like as is known in the art for such system for choosing apparel, and implementing components and methods, consistent with the intended operation and methods.

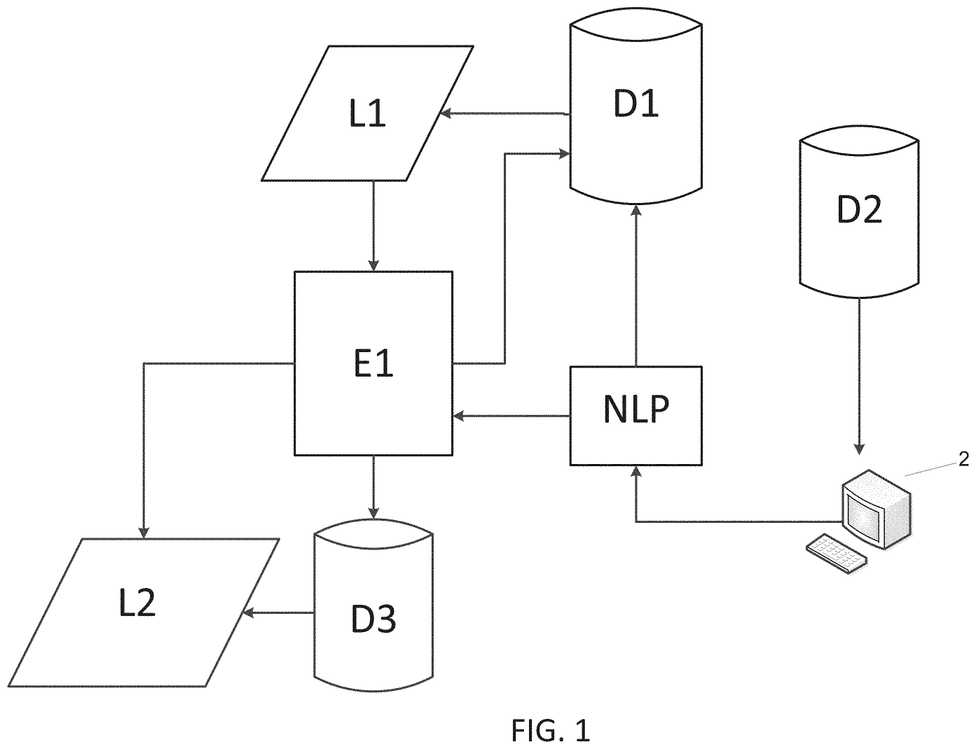

Referring to FIG. 1, a schematic view of a system for selecting clothing and apparel based on medical events and needs are illustrated. As described herein, medical events may include surgeries, ongoing treatment plans, chronic illnesses, or other health related challenges. Specific examples of medical events or health related challenges may include, by non-limiting example, surgery, cancer, multiple sclerosis (MS), ostomies, medications administered by pumps, strokes, diabetes, decompensation, any combination thereof, or any other medical event than may require clothing that accommodates medical devices, reduces range of motion, or in some way impairs a person's activities of daily life. Choosing clothing may be difficult for the individual because of health-related criteria such as skin sensitivity, isolated swelling, temperature sensitivity, weight gain, fluid retention, and other complications that may arise from health-related challenges.

As illustrated, the system includes a first database D1. The first database includes combinations of medical events and challenges, the physical limitations or impairments caused by the medical challenges, and recommendations for apparel characteristics to accommodate the physical limitations of the medical challenges or impairments. The first database is initially populated by expert medical advice. Those in the medical field may include doctors, nurses, occupational therapists, physical therapists, home health aides, and others who may assist individuals with health related challenges often suggest clothing articles that are compatible with the health related challenge of the individual.

The first database is configured to increase in size such as increasing the total number combinations, specifications based on the medical challenges and preferences of the users. As described herein, the user of the system may include a person facing a medical event or challenge. In other implementations, the user may be referred to as a patient, a client, a resident, and other terms used in the medical community to refer to a person under the care of a medical professional. In still other implementations, the user may include a family member or friend who may interact with the system to select apparel items for a beneficiary user that is facing a medical challenge or event. By non-limiting example, the database may initially include an unlimited combination of apparel items, medical challenges, and medical events, with these combinations being supplied by a medical professional. In various implementations, a medical professional may also be referred to as a medical expert. As the system for choosing apparel items is used in various implementations of methods for choosing apparel, the size and personalization of the combinations will increase. As the sample size increasing with an increasing number of consumers, the personalization and the automation of the system will increase. In about a year, the amount of combinations per medical challenge will increase to at least 100 combinations for a total of 1,000 recommendations stored within the first database. In some implementations, the total number of recommendations stored within the first database may increase to over 1,000 recommendations.

Still referring to FIG. 1, the system also includes a second database D2. The second database includes various questions that may be sent to a user. In various implementations, two or more questions may be sent to a user. The two or more questions may be sent in the form of a questionnaire. In various implementations, the questions may include selecting an answer from multiple choices such as age, sex, location, or other demographic information. In some implementations, the answers may be selected from a dropdown menu. In other implementations, the answers may be selected by checking one or more boxes. The questions may also include open-ended questions with a space to provide a free form answer. The open-ended questions may allow a user to answer the questions naturally without having to worry about correct terminology or whether their preferences are listed as choices. As illustrated, the questions will be sent to the user through a user interface 2. Here, a desktop computer is illustrated. In various implementations, the user interface may include a cellular phone, a tablet, a laptop computer, or any other device used to access telecommunication channels that allow a user to enter information. In some implementations, the user may enter information through text, typing, voice commands, or other methods of entering information into a personal computer device.

The system for choosing clothing and apparel based on medical events and challenges also includes a natural language processor (NLP). The NLP may also gather information about the user through the free text response answers given to the open ended questions. In various implementations, the NLP may process transcripts of voice responses to the questions. The NLP extracts semantic primitives from the answers in order to determine the medical event or challenge the user is experiencing. Semantic primitives are a set of language-agnostic concepts that are innately understood but cannot be expressed in simpler terms. Semantic primitives are concepts that are learned through practice and that may have difference expressions as words or phrases across differing languages, and that are learned through practice but cannot be defined concretely. The NLP may also extract semantic primitives to extract the preferences of the user regarding size, colors, brands, price point, and other preferences associated with apparel and clothing. The NLP may also extract details about the medical challenge or event such as the user having a limited range of motion, being unable to bend over, needing clothing to accommodate medical devices, and other clothing attributes associated with medical challenges and events.

Still referring to FIG. 1, the system for choosing apparel based on medical events and challenges also includes a rules engine E1. The rules engine may use an algorithm including a forward-chaining rules engine that implements a fuzzy logic calculation based on a Bayes' Theorem to produce the personalized list of one or more recommended apparel items. Forward chaining or forward reasoning is one of the two main methods of reasoning when using an inference engine. It can be described logically as repeated application of modus ponens. Modus ponens is a rule of inference that can be summarized as “P implies Q and P is asserted to be true, therefore Q must be true.” Forward chaining starts with the available data and uses inference rules to extract more data until a goal is reached. In particular implementations of a system for choosing apparel, the rules engine will continue to collect data from user input and produce more recommendations for a plurality of users each of whom may be experiencing one of a plurality of medical events or challenges. The algorithm of the rules engine also uses a fuzzy logic which is a form of many-valued logic in which the truth values of variables may be any real number between 0 and 1 inclusive. The algorithm also includes an application of Bayes' Theorem, which describes the probability of an event, based on prior knowledge of conditions that might be related to the event. The algorithm employed by the rules engine may be able to provide continually more personalized recommendations as the first database continues to be updated.

The system for making apparel recommendations also includes a third database D3. The third database includes one or more retailers of a plurality of apparel characteristics with each of a plurality of apparel items recommended for medical events based on health related criteria. The system may allow a user to get the information of working directly with a personal shopper over the internet. Various apparel items may be recommended to the user based on the information provided by the user in the free text response. The system may also allow a user to get expert medical advice on the various apparel characteristics need in a plurality of apparel items while facing experiencing a medical event or challenge. The system may free up valuable resources of the medical professionals who respond to phone calls and emails asking for clothing recommendations from patients and clients. The system also is able to make recommendations based on the preferences of the user combined with the apparel characteristics needed during various health related challenges. Therefore, the system is able to combine the expertise of a medical professional with the expertise of personal shopper that is available to a user any time of the day or night rather than only during business hours. By including the third database with a plurality of retailers having a plurality of apparel characteristics with each of a plurality of apparel items, a user is not confined to a single retailer or brand as might be the case with a personal shopper.

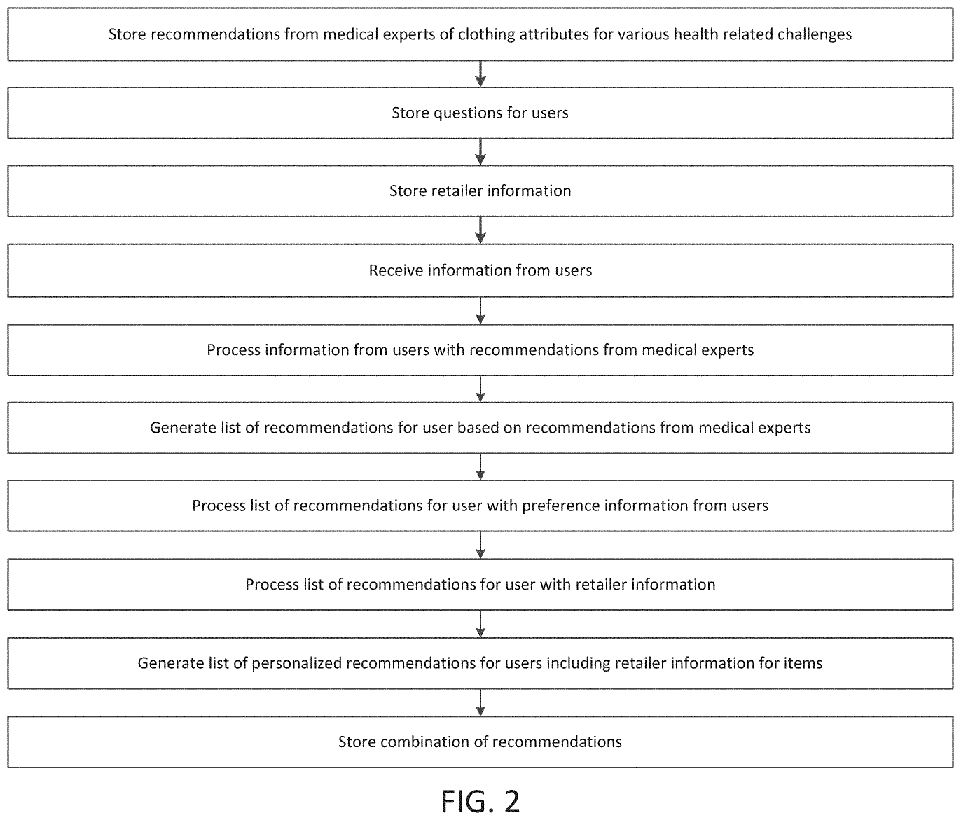

Referring to FIG. 2, a method of building a database of apparel recommendations may be performed using an implementation of an automated system for making apparel recommendations. The method may include storing a plurality of apparel characteristics with each of a plurality of apparel items recommended for medical events based on information from one or more medical professionals. The plurality of apparel characteristics with each of the plurality of apparel items may be stored in the first database D1. The method may also include storing questions for a plurality of users in the second database D2. Each of the plurality of users may be experiencing one or more of a plurality of medical events when answering the questions in the questionnaire. The questions may request information about the user including medical challenges and events the user is facing, preferences in brands, sizes, material, location, age, and other information that may influence the choosing of apparel. At least one of the questions may be designed to accept a free text response. As illustrated, the method includes sending two or more questions to the users and receiving information from the users. The information may be received in the form of two or more questions that are sent through a telecommunication channel to a computing device associated with a user. The computing device associated with the user may be configured to generate a user interface include the two or more questions in response to receiving the two or more questions.

Still referring to FIG. 2, the method also includes processing the two or more answers from the plurality of user using a natural language processor NLP. As previously described, the natural language processor may be able to extract semantic primitives from the free text in response to the two or more questions. By extracting semantic primitives, the NLP is able to determine what a user types in the free text responses without the user needing to worry about how they are describing things. In various methods of building a database of apparel recommendations, the NLP may extract one or more medical events of each of the plurality of users and one or more preferences of each of the plurality of users from the two or more answers from a plurality of users. The method also includes generating a list of recommended apparel items for each of the plurality of users based on the one or more medical events extracted from the answers to the two or more questions received from the computing device. The method includes generating the list of recommended apparel items using the first database D1 and the rules engine E1. The rules engine may use the algorithm including forward chaining, fuzzy logic, and Bayes' Theorem to calculate characteristics of apparel items and apparel items that are compatible with various medical challenges.

The method also includes processing the list of recommended apparel items and the one or more preferences of each of each of the plurality of users to form a list or preferred recommended apparel items. The list may be processed using the rules engine E1, the first database D1 and the third database D3. The method further includes generating with the list of preferred recommended apparel items and the third database a personalized list of recommended apparel items for each of the plurality of users. The personalized list may include one or more recommended items based on one or more criteria including a health challenge of user, one or more size preferences of the user, one or more color preferences of the user, one or more geographical locations of user, or any combination thereof. The criteria may be extracted from the two or more answers to the two questions in the user interface. The method also includes adding the personalized list of recommended apparel items for each of the plurality of users to the first database D1. The method may further include an updating process that continually updates the first database of apparel characteristics with each of a plurality apparel items recommended for medical events based on health related criteria. Therefore, each personalized list is stored in the first database D1 and the size and personalization abilities of the first database D1 may be increased through machine learning.

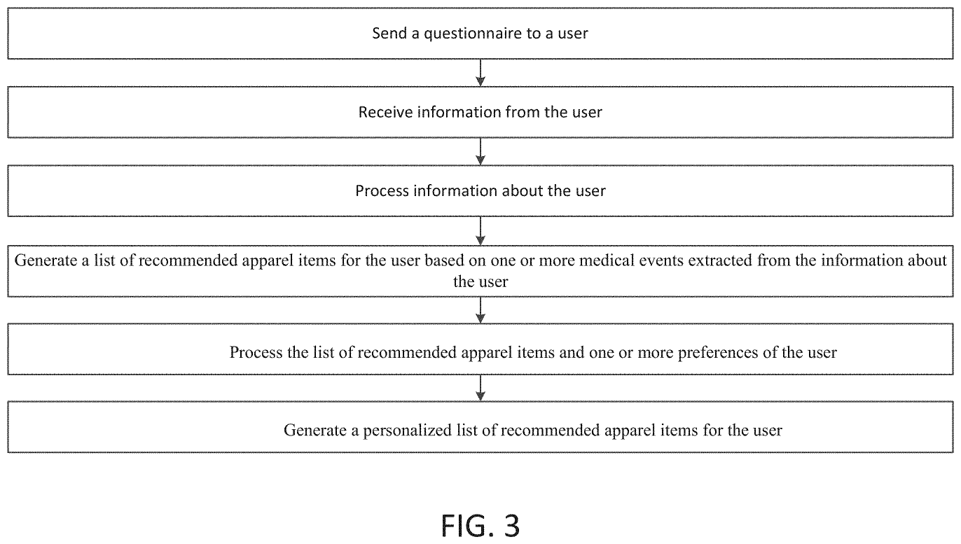

Referring to FIG. 3, an implementation of an automated method for selecting apparel is illustrated. Through not illustrated, the method may include selecting a user facing a medical event. In various implementations, the user may be self-selected, selected by a friend, family member, co-worker, medical professional, or any person with knowledge of the user experiencing a medical challenge or event. In some implementations of the automated method, the user may be a person related to the person with the medical challenge. In such implementations, the person with the medical challenge may be referred to a beneficiary user. The method includes sending a questionnaire to a computing device associated with the user. In various implementations, the computing device may include a desktop computer, a laptop, a tablet, a cell phone, or any computing device capable of allowing communication over the internet or other telecommunication channels. The computing device may be configured to generate a user interface including a questionnaire. The questionnaire may be sent through a telecommunication channel. In various implementations, the telecommunication channel may be any described herein.

Referring again to FIG. 3, the method includes receiving information about the user. The information may include two or more answers to the questionnaire sent to the user via the computing device. The two or more answers may be processed using a natural language processor. The information may include one or more medical challenges experienced by the user, and one or more preferences of the user including color, brand, fabric, size, price points and other clothing characteristics. The natural language processor may extract the information from the answers using semantic primitives.

The method also includes generating a list of recommended apparel characteristics with each of a plurality of apparel items for the user using the one or more medical events extracted from the two or more answers. The list of recommended apparel characteristics may be generated using the first database. The method then includes processing the list of recommended apparel items and the one or more preferences of the user to generate a preferred recommended list for the user. A personalized list of recommended apparel items may be generated using the rules engine and a third database of retailers.

The automated method for selecting apparel may include communicating to the computing device the personalized list of items using the computing device generated user interface including a personalized list of recommended apparel items. In some implementations, the notification may include an email, a text, an alert, and other methods of notifying a person through a computing device. The information may be communicated over a telecommunication channel. The method also include sending the personalized list or one or more items from the personalized list to one or more preselected potential buyers of one or more items. In various implementations, the user, the beneficiary user, and from the personalized list may be notified. The method may further include sending a beneficiary user a unique identifier of a beneficiary user interface to notify the beneficiary user of the beneficiary user interface. In various implementations, the beneficiary user may be sent a unique identifier through an email or mailed on a postcard. The method may further include facilitating the purchase of a personalized item through the third database of apparel retailers. In some implementations, an organizer may send an item to a plurality of preselected buyers allowing them to contribute an amount that is less than a total purchase price of an item.

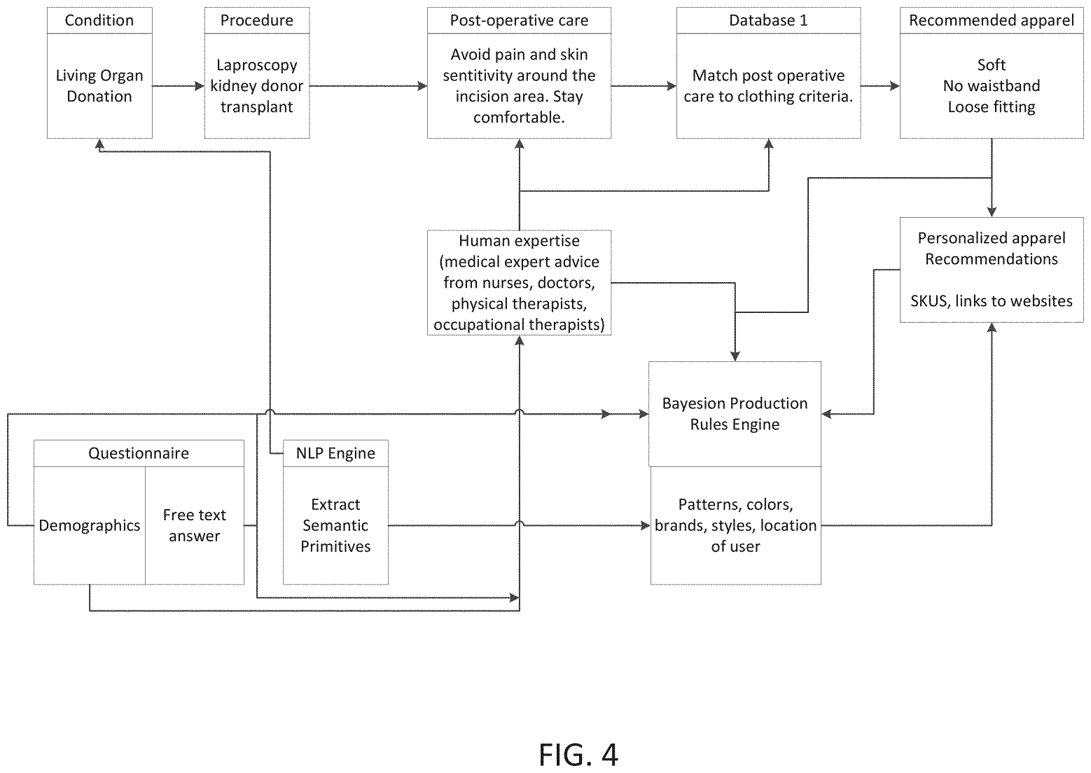

Referring to FIG. 4, a high-level implementation of a method of choosing apparel for a medical event is illustrated. This particular implementation includes a user experiencing a live organ donation. FIG. 4 includes what the user may see on the user interface such as the questionnaire and personalized recommendations. FIG. 4 also includes the elements of the system that a user will not see such as the natural language processor, the rules engine, and the first database.

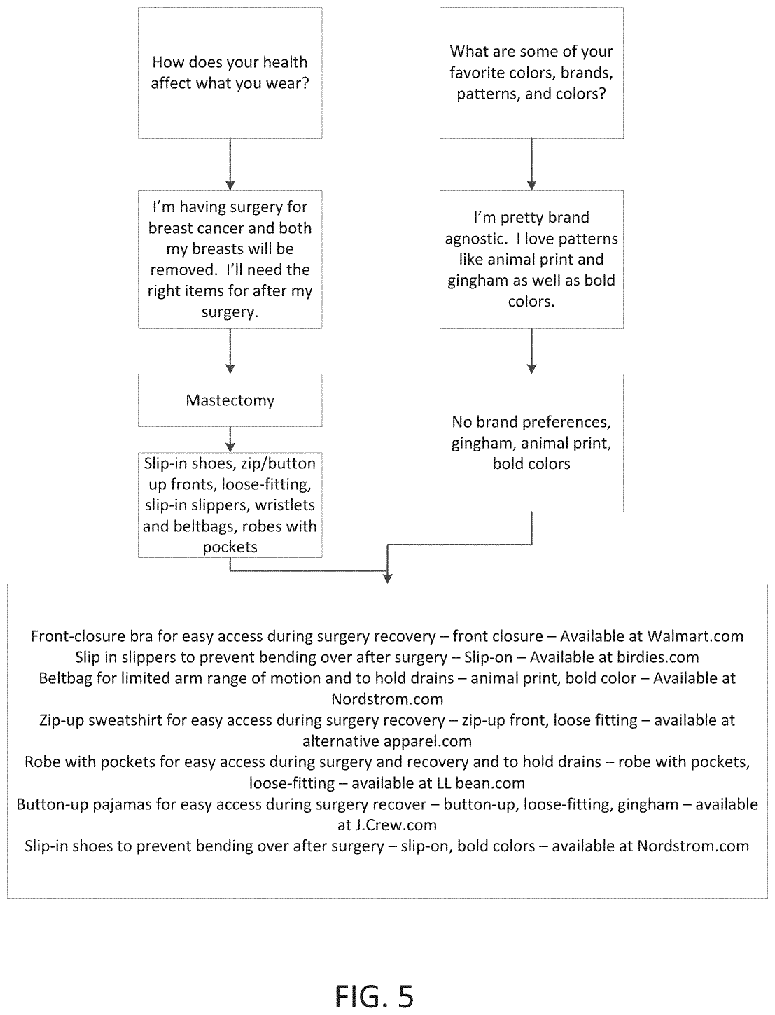

Referring to FIGS. 5-8, detailed examples of implementations of a method of choosing apparel for a medical event are illustrated. In these figures, a combination of user interface and other elements of the system are illustrated. For example FIG. 5 illustrates, the questions the user will see as well as the answers to the two questions. The information is extracted from the free text and used to generate a personalized list of recommendations. The example in FIG. 5 demonstrates a user experiencing a mastectomy. Though not illustrated, this user may also experience other medical events such as cancer that may or may not be included in the calculations or recommendations.

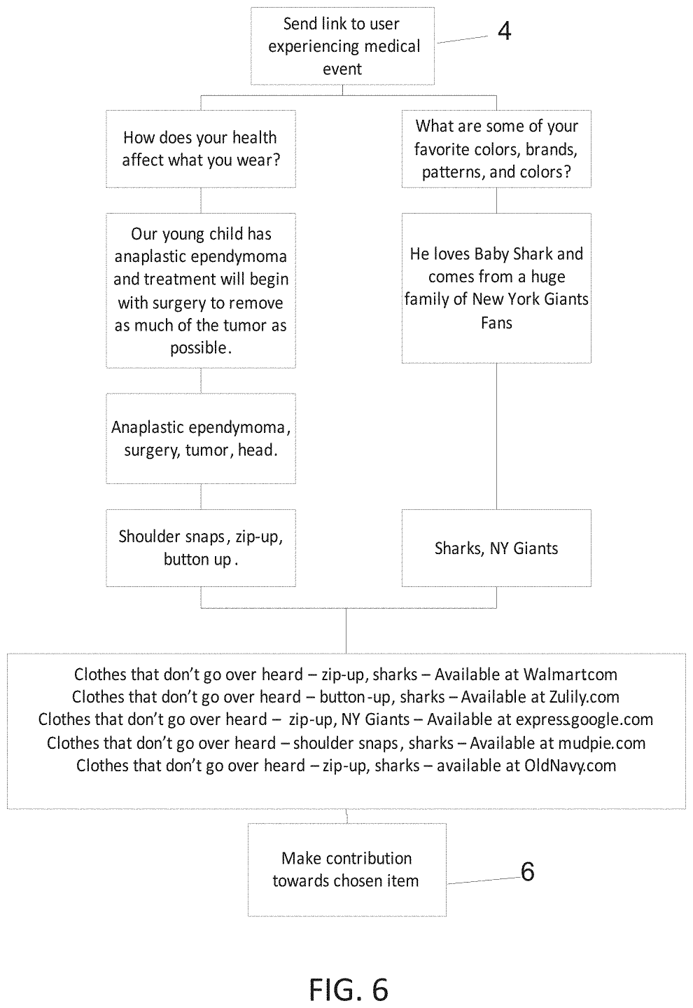

Referring to FIG. 6, a detailed example of method for choosing apparel for a medical event is illustrated. As illustrated by the first box 4 of the flowchart, this particular implementation includes a user and a beneficiary user. The user may enter initial information about the beneficiary user into a user interface and a link may be sent to the beneficiary user to answer the free text response questions. In some implementations, the user may answer the questions on behalf of the beneficiary user. Such scenarios include a parent answering the questions for a child, a spouse answering the questions for another spouse, an adult child answering the questions for an aging parent, and other caregiver scenarios. This implementation also illustrates sending the personalized list to one or more preselected potential buyers 6 in order to allow them to contribute to one or more recommended items.

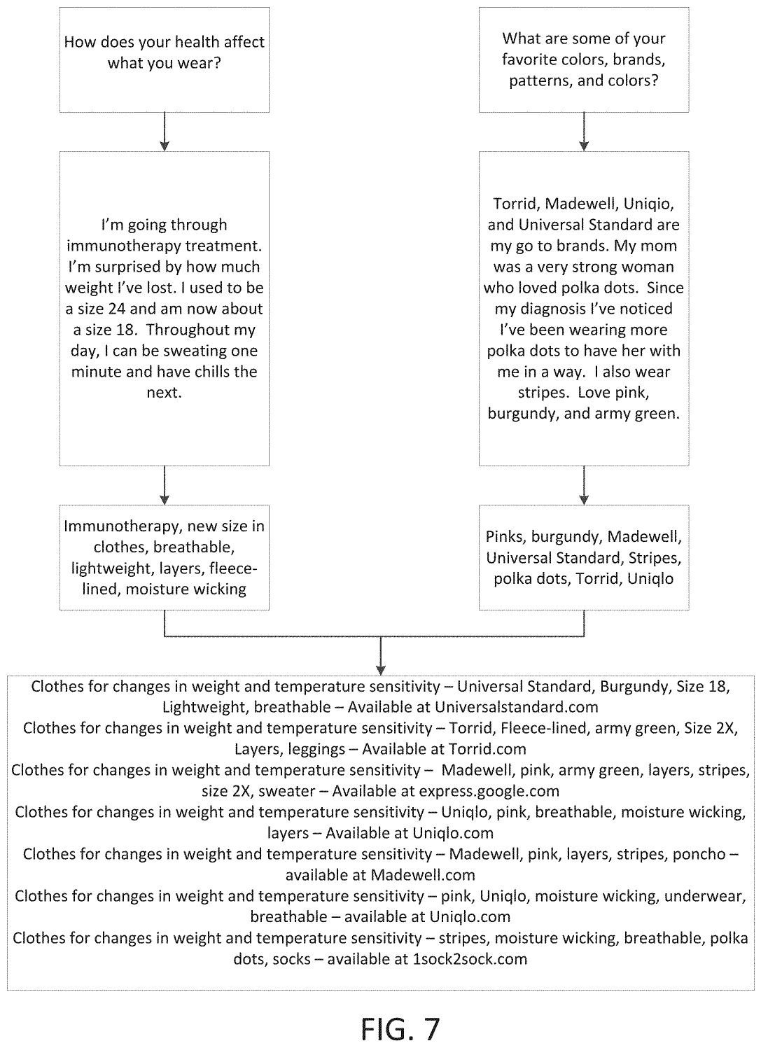

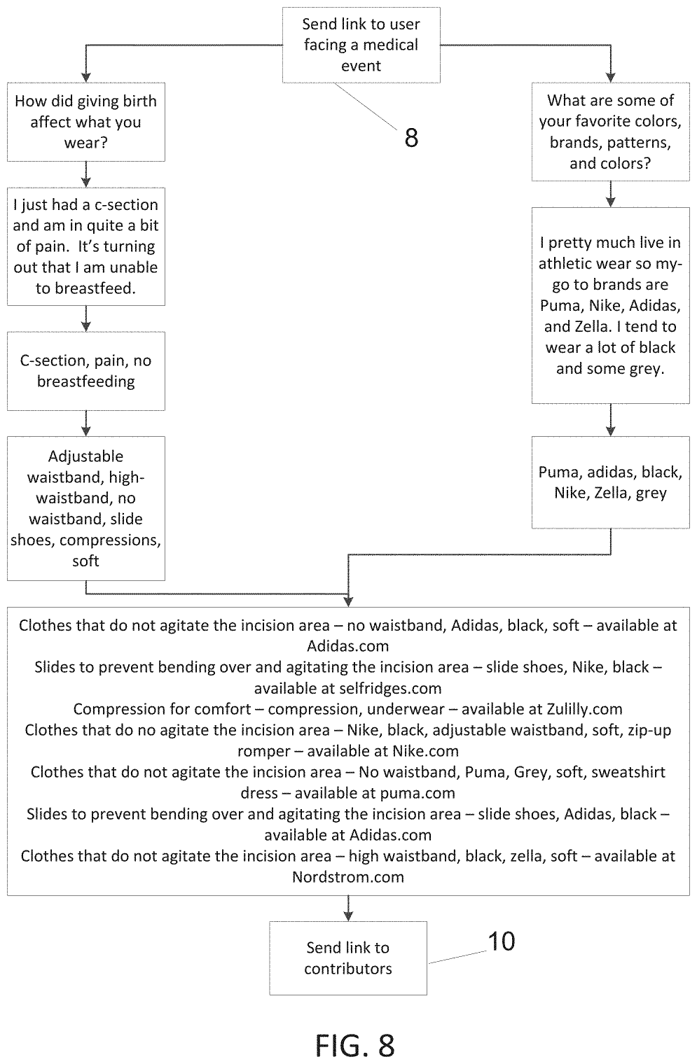

Referring to FIG. 7, another detailed example of method for choosing apparel for a medical event is illustrated. The user's answers to the two or more questions are illustrated and a personalized list or recommended items are generated by the system. Referring to FIG. 8, another example of a user sending the questionnaire 8 to a beneficiary user is illustrated. The personalized list may be communicated to one or more potential buyers 10 through a user interface on a computing device.

In places where the description above refers to particular implementations of systems for choosing apparel and implementing components, sub-components, methods and sub-methods, it should be readily apparent that a number of modifications may be made without departing from the spirit thereof and that these implementations, implementing components, sub-components, methods and sub-methods may be applied to other automated systems for choosing apparel.

What is claimed is:

1. An automated system for making apparel recommendations:
a first database comprising a plurality of apparel characteristics with each of a plurality of apparel items recommended for medical events based on health related criteria;
a second database comprising two or more questions requesting information about the user, wherein the two or more questions are configured to be displayed on a user interface of a computing device, at least one of the questions designed to accept a free text response;
a natural language processor configured to extract semantic primitives from two or more answers to the two or more questions from the free text portion of the user interface;
a third database of one or more retailers of a plurality of apparel characteristics with each of a plurality of apparel items recommended for medical events based on health related criteria; and
a rules engine configured to use the semantic primitives from the natural language processor, the first database, and the third database to produce a personalized list of one or more recommended apparel items for the user who has experienced a specific medical event.

2. The system of claim 1, wherein the first database comprises apparel items by expert medical recommendations.

3. The system of claim 1, wherein the rules engine comprises an updating process that continually updates the first database of apparel characteristics with each of a plurality apparel items recommended for medical events based on health related criteria.

4. The system of claim 1, wherein the rules engine uses an algorithm comprising a forward-chaining rules engine that implements a fuzzy logic calculation based on a Bayes' Theorem to produce the personalized list of one or more recommended apparel items.

5. The system of claim 1, wherein the personalized list comprises recommended items based on one or more criteria including a health challenge of the user, one or more size preferences of the user, one or more color preferences of the user, one or more brand preferences of the user, one or more geographical locations of the user, or any combination thereof, these criteria extracted from the two or more answers to the two questions in the user interface.

6. The system of claim 1, wherein the natural language processor is configured to extract semantic primitives from free text responses or voice-to-text transcripts.

7. A method of building a database of apparel recommendations, the method comprising:
storing, in a first database, a plurality of apparel characteristics with each of a plurality of apparel items recommended for medical events based on information from one or more medical professionals;
storing, in a second database, two or more questions for a plurality of users, each user experiencing one or more of a plurality of medical events;
sending, through a telecommunication channel, to a computing device associated with a user, the two or more questions from the second database to the plurality of users, the computing device associated with the user configured to generate a user interface comprising the two or more questions in response to receiving the two or more questions;
receiving from the computing device, through a telecommunication channel, two or more answers to the two or more questions from the user interface;
processing, with a natural language processor, the two or more answers from the plurality of users to extract the one or more medical events of each of the plurality of users and one or more preferences of each of the plurality of users;
generating, using the first database and the rules engine, a list of recommended apparel items for each of the plurality of users based on the one or more medical events extracted from the answers to the two or more questions received from the computing device;
processing, using a third database of apparel retailers and the rules engine, the list of recommended apparel items and the one or more preferences of each of the plurality of users to form a list of preferred recommended apparel items;
generating with the list of the preferred recommended apparel items and the third database of apparel retailers, using one or more calculations of the rules engine, a personalized list of recommended apparel items for each of the plurality of users; and
adding, the personalized list of recommended apparel items for each of the plurality of users to the first database.

8. The method of claim 7, wherein a size of the first database is increased through machine learning.

9. The method of claim 7, wherein the second database comprises at least one of a demographic question and a free text entry question.

10. The method of claim 7, wherein the rules engine uses an algorithm comprising a forward-chaining rules engine that implements a fuzzy logic calculation based on Bayes' theorem to produce the personalized list of one or more recommended apparel items.

11. The method of claim 7, wherein the personalized list comprises one or recommended items based on one or more criteria including a health challenge of the user, one or more size preferences of the user, one or more color preferences of the user, one or more brand preferences of the user, one or more geographical locations of the user, or any combination thereof, these criteria extracted from the two or more answers to the two questions in the user interface.

12. The method of claim 7, wherein the natural language processor is configured to extract semantic primitives from free text responses or voice-to-text transcripts.

13. An automated method for selecting apparel, the method comprising:
selecting a user facing a medical event;
sending, through a telecommunication channel, a questionnaire to a computing device associated with the user the computing device configured to generate a user interface comprising the questionnaire, the questionnaire using a second database comprising two or more questions;
receiving, through a telecommunication channel, two or more answers to the questionnaire from a user via the computing device;
processing, with a natural language processor, the two or more answers from the user to extract one or more medical event of the user and one or more preferences of the user;
generating, using the first database, a list of recommended apparel characteristics with each of a plurality of apparel items for the user using one or more medical events extracted from the two or more answers;
processing, using a rules engine, the list of recommended apparel items and the one or more preferences of the user; preferred recommended
generating, using the rules engine and a third database of retailers, a personalized list of recommended apparel items;
communicating, through a telecommunication channel, to the computing device the personalized list of items using the computing device generated user interface comprising a personalized list of recommended apparel items; and
sending, using the user interface of the computing device, to one or more preselected potential buyers one or more items from the personalized list.

14. The method of claim 13, wherein the user comprises one of a person dealing with a medical event, a friend, a family member, a medical professional, a social worker, or any combination thereof.

15. The method of claim 13, wherein the rules engine uses an algorithm comprising a forward-chaining rules engine that implements a fuzzy logic calculation based on a Bayes' Theorem to produce the personalized list of one or more recommended apparel items.

16. The method of claim 13, wherein the personalized list comprises recommended apparel characteristics based on one or more criteria including a health challenge of the user, one or more size preferences of the user, one or more color preferences of the user, one or more brand preferences of the user, one or more geographical locations of the user, or any combination thereof, these criteria extracted from the two or more answers to the two questions in the user interface.

17. The method of claim 13, wherein the natural language processor is configured to extract semantic primitives from free text responses or voice-to-text transcripts.

18. The method of claim 13, further comprising sending a beneficiary user of the user a unique identifier of a beneficiary user interface to notify the beneficiary user of the beneficiary user interface.

19. The method of claim 18, wherein sending the beneficiary user a unique identifier comprises one of sending an email or sending a postcard.

20. The method of claim 13, further comprising facilitating the purchase of a personalized item through a third database of apparel retailers.
